(19) United States
(12) Patent Application Publication
(10) Pub. No.: US 2012/0089152 A1
(43) Pub. Date: Apr. 12, 2012

(54) ANIMAL TAG APPLICATOR

(75) Inventors: Malcolm Norman Lynd, Auckland (NZ); Roy Victor Bladen, Auckland (NZ); Michael Stuart Gardner, Auckland (NZ)

(73) Assignee: TAGAM LIMITED, Auckland (NZ)

(21) Appl. No.: 13/376,190

(22) PCT Filed: May 14, 2010
(86) PCT No.: PCT/NZ2010/000090

§ 371 Date: Dec. 4, 2011

(30) Foreign Application Priority Data
Jun. 3, 2009 (NZ) 577476

(51) Int. Cl. A61B 17/00

(52) U.S. Cl. 606/117

(57) ABSTRACT

An animal tag applicator (1) has a rear body portion (4) slidable forwardly over a front body portion (3) to load a frontmost tag (10) between first and second jaws (7), (8) from a strip of tags (not shown) accommodated longitudinally within a chamber defined by the body portions (3), (4). Operation of a handle (5) against its bias (12) can then effect a tagging operation after which further forward movement of the body portion (4) will re-load the applicator (1) ready for the next tagging operation.

### FIELD OF THE INVENTION

This invention relates to improvements in relating to animal tag applicators.

More particularly, but not exclusively, the present invention relates to applicators which are suitable for farm animals such as sheep.

### BACKGROUND TO THE INVENTION

To the present time, many tag applicators are able to be used solely for the application of a single tag to an animal, typically to its ear. Such applicators require the loading of discrete tags ready for each application with the consequential time and effort that this involves. Other applicators, however, have proposed the use of a strip of tags which can be loaded into the applicator so that tags can be sequentially dispensed from the applicator.

Previous proposals have suffered from various disadvantages both in terms of ease of operation and reliability. For example, in U.S. Pat. No. 5,234,440, a strip of tags is loaded from the side of the applicator. A leading tag is then severed from the tag strip to be advanced to the front of the applicator. Such side loading, and the early separation of the tag from the strip, both result in problems during use. Other proposals using the side loading of the strip of tags have a further disadvantage that the first two tags of the strip are unusable, their role solely being to assist in the loading operation, see, for example, New Zealand patent 524953.

In contrast, in our own WO 2008/075974, an applicator is described in which the strip of tags is loaded and subsequently fed along the longitudinal direction of the body of the applicator. The operation of a spring loaded handle results in the movement of the frontmost tag into the loaded position and its subsequent engagement about the animal's ear, before release and the subsequent loading into the position of the next tag, so that the tagging operation can continue. Although having many advantages over earlier proposals, such as those mentioned above, the need to continually operate the spring loaded handle of that applicator, for both the loading and tagging operation, can be tiring when a large number of animals are required to be tagged.

### OBJECT OF THE INVENTION

It is an object of at least preferred embodiments of the invention to provide an animal tag applicator which will at least obviate difficulties in previous applicators, or which will at least provide the public with a useful choice.

Further objects of the invention will become apparent from the following description:

### BRIEF SUMMARY OF THE INVENTION

According to one aspect of the present invention there is provided an animal tag applicator including an applicator body having at least first and second body portions defining a chamber adapted, in use, to accommodate a strip of tags substantially along a longitudinal axis of said body, said second body portion being moveable substantially in a direction of said longitudinal axis relative to said first body portion whereby a frontmost tag of the strip of tags, in use, is located in a tagging position, an operating means moveable relative to said first body portion and adapted, in use, to engage said frontmost tag for it to be secured about a part of an animal in a tagging operation.

Preferably, the second body portion is adapted to move in a forward direction towards a front end of said first body portion in moving said frontmost tag, in use, to said tagging position and subsequently to move in a rearward direction away from said front end of said first body portion before movement of the next tag into the tagging position.

Preferably, the second body portion is adapted to slide over said first body portion and in said forward and rearward movement.

Preferably, said operating means includes a handle pivotally mounted relative to said first body portion.

Preferably, said handle is biased into an open position away from said first body portion.

Preferably, said handle includes at a front end thereof, a first jaw portion movable towards a second jaw portion in engaging with said frontmost tag to provide said tagging operation.

Preferably, the tags have male and female portions which are engaged together after said frontmost tag is engaged.

Preferably, said first jaw portion includes a cutting means adapted to engage with and cut a rear portion of said frontmost tag in separating the frontmost tag from a next adjacent tag prior to or during a tagging operation.

According to a second aspect of the present invention there is provided an applicator substantially as herein described with reference to any one of the embodiments of the invention and with reference to the accompanying drawings.

According to a third aspect of the present invention there is provided a strip of tags adapted to be used in the applicator of any one of the nine immediately above paragraphs.

Further aspects of this invention, which should be considered in all its novel aspects, will become apparent from the following description, given by way of example of possible embodiments thereof, and in which reference is made to the accompanying drawings.

### BRIEF DESCRIPTION OF THE DRAWINGS

FIG. 1: Shows a side perspective view of an applicator according to one possible embodiment of the invention in a loaded condition with a frontmost tag ready for a tagging operation to take place;

FIG. 2: Shows an underneath perspective view of FIG. 1;

FIG. 3: Shows a side view of FIG. 1;

FIG. 4: Shows the applicator with the male and female portions of the frontmost tag engaged together as part of a tagging operation;

FIG. 5: Shows a plan perspective view of FIG. 4 but with the top jaw portion having being released from engagement with the frontmost tag;

FIG. 6: Shows a side view of FIG. 5;

FIG. 7: Shows a side perspective view of the applicator following a tagging operation and a tag in position about an animal's ear;

FIGS. 8(a) and (b): Show respectively and diagrammatically, in part cross-section, an applicator according to one possible embodiment of the invention about to be loaded with the strip of tags and a plan view of FIG. 8(a);

FIG. 9: Shows a cross-sectional view of an applicator loaded with a strip of tags but without a frontmost tag in the tagging position;

FIG. 10: Shows the applicator of FIG. 9 after the forward sliding of the rear body portion of the applicator has moved the strip of tags forwardly and loaded a frontmost tag into the tagging position;

FIG. 11: Shows the applicator of FIG. 10 with the sliding body portion returned to its rearward position and the handle compressed, causing the closure of the frontmost tag;

FIG. 12: Shows the applicator of FIG. 11 with the frontmost tag having its male and female portions fully engaged with the animal's ear (not shown);

FIG. 13: Shows the applicator of FIG. 12 with the tagged animal's ear withdrawn from the applicator (ear not being shown);

FIG. 14: Shows in cross-section the applicator according to one possible embodiment of the invention and with greater detail shown of the cutting means provided by the upper jaw portion;

FIG. 15: Shows the applicator of FIG. 14 with the jaw portions closed and the cutting means operated to sever the frontmost tag from the second most tag (the tags not being shown);

FIGS. 16(a), (b), (c) & (d): Show respectively and diagrammatically, side, plan, underneath and end views of an applicator according to one possible embodiment of the invention in its storage position.

### BRIEF DESCRIPTION OF POSSIBLE EMBODIMENTS

As mentioned above, in our earlier applicator of WO 2008/075974, for the sequential movement of tags through the applicator and their sequential tagging on an animal's ear, both operations rely on the operation of a spring biased handle. After a substantial number of tagging operations, an operator can be become tired due to the continual compression of the handle against the spring bias. In contrast, in the present invention, the sequential movement of the tags of a strip of tags can be simply and easily achieved, preferably by a sliding action, so that, as will become clear, it is only the tagging operation itself which requires the compression of a handle.

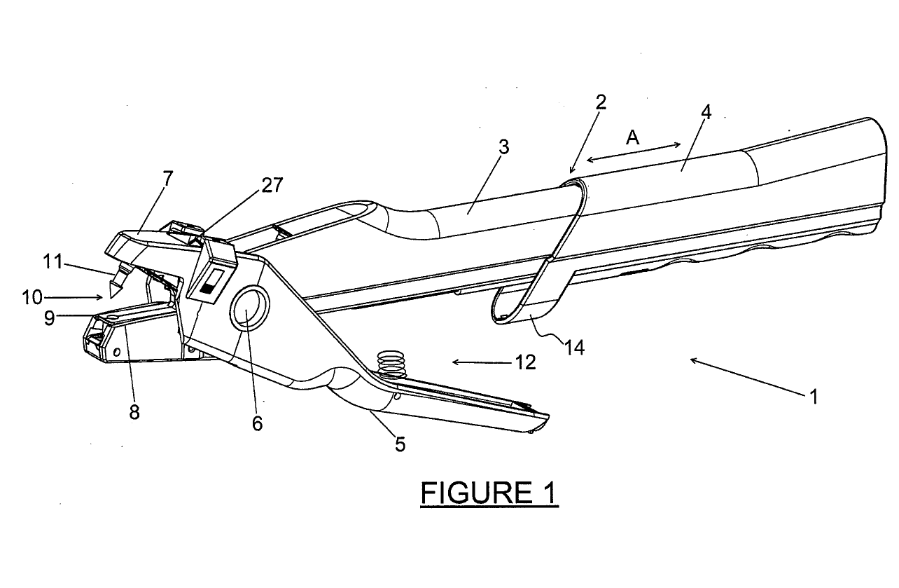

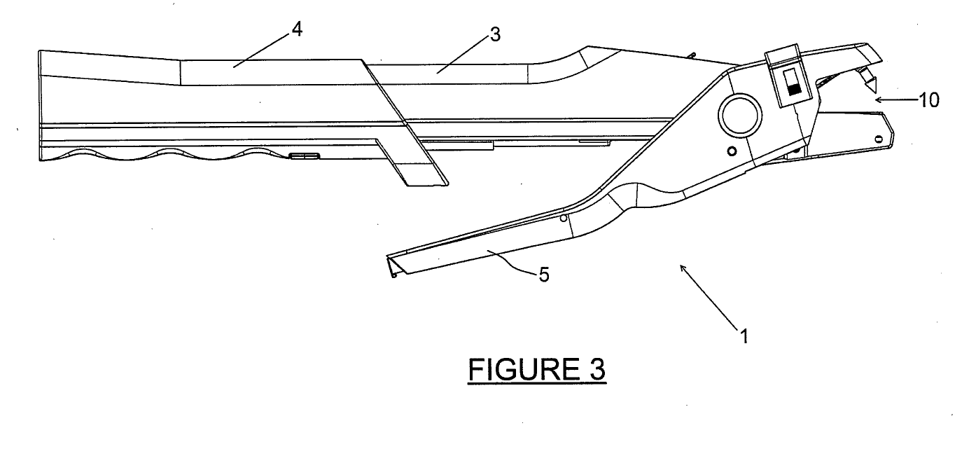

Turning therefore to the accompanying drawings, and firstly to FIGS. 1 to 3, an applicator is referenced generally by arrow 1 and is shown having a body portion referenced generally by arrow 2 which includes a first front body portion 3 over which a second rear body portion 4 is able to slide in forward and rearward directions indicated by arrows A. Handle 5 is shown pivoted at 6 towards the front of the body portion 3 and is provided with a first top jaw portion 7 which, on the pivoting of the handle 5, can move towards a second lower jaw portion 8 at a front end of the first body portion 3 and on which is shown positioned in FIG. 1, the female portion 9 of a frontmost loop tag, indicated by arrow 10. As shown, the male portion 11 of the tag 10 has been engaged by the top jaw portion 7 to force the male portion 11 towards the female portion 9 in order to tag the animal's ear (not shown).

In an alternative embodiment, the jaw portions 7 and 8 may be suitably adapted for the tagging of one piece tags, other than a loop tag as shown, or two piece tags.

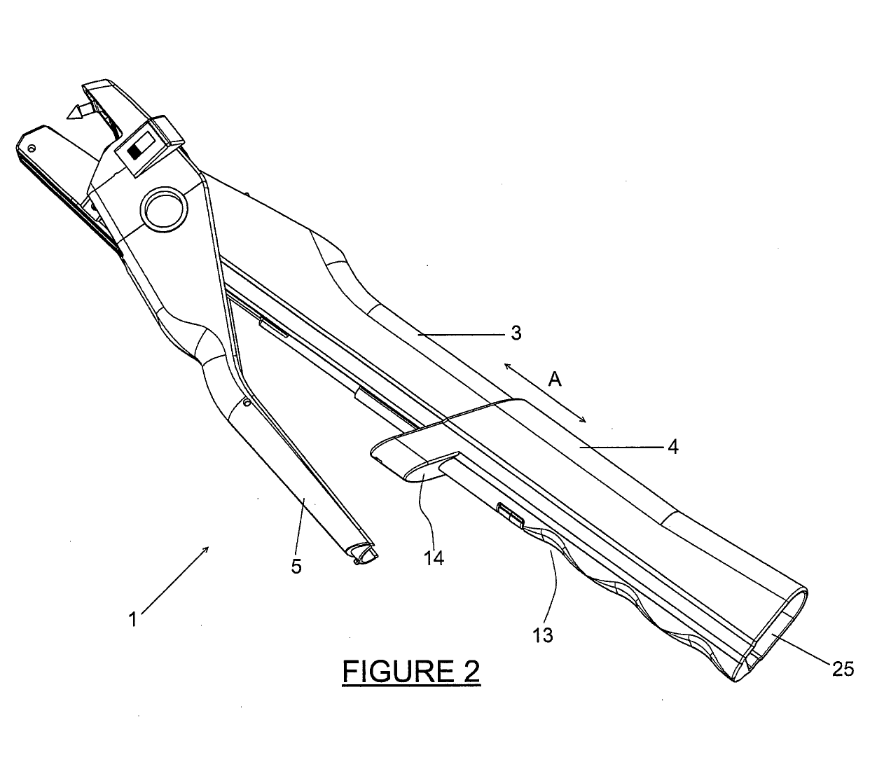

As indicated very diagrammatically by arrow 12, a suitable biasing means, such as a compression spring, may bias the handle 5 into its open position away from the body portion 3, so that operation of the handle 5 during the tagging operation is against the spring or other bias. As seen particularly in FIG. 2, the slidable body portion 4 may have finger holds or the like 13 and a front end projection 14 so that the body portion 4 can be readily gripped by a user in being slid forwardly and rearwardly as indicated by the arrows A relative to the front body portion 3. Again, as shown particularly in FIG. 2, the rear end 25 of the body portion 4, may be open so as to readily permit the introduction therethrough of the strip of tags 10 when the applicator 1 is loaded.

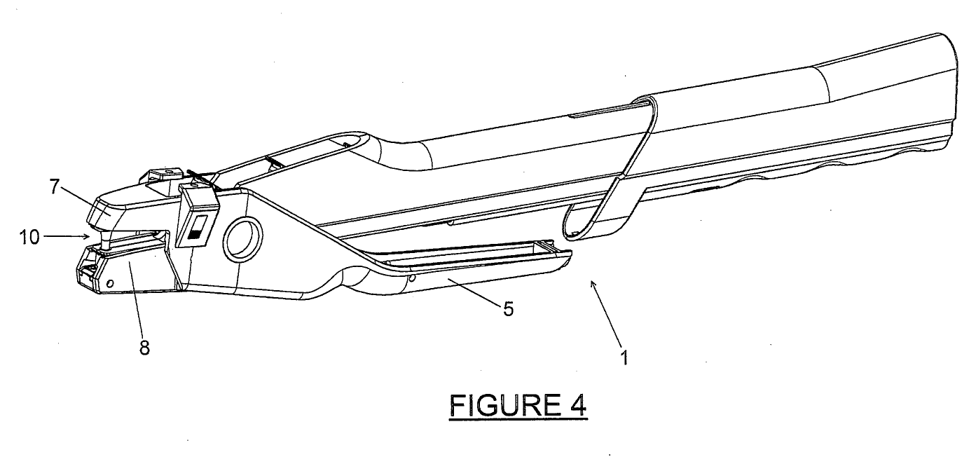

Turning then to FIG. 4, in this position, the handle 5 has been closed against the spring or other bias, (see FIG. 1), bringing the jaw portions 7 and 8 closer together and compressing the male and female portions of the tag 10 so that they engage about the animal's ear (not shown).

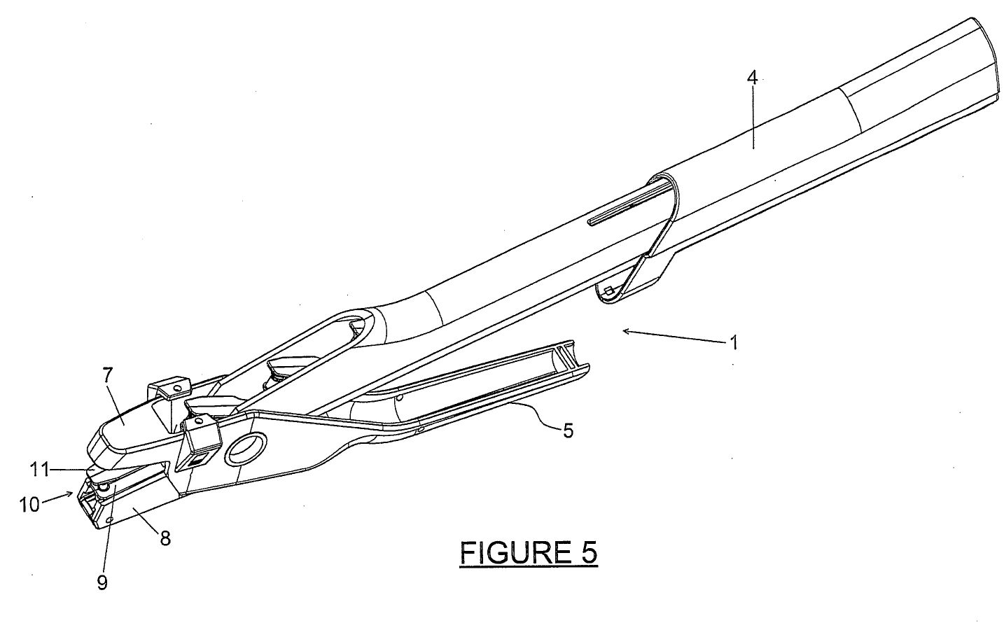

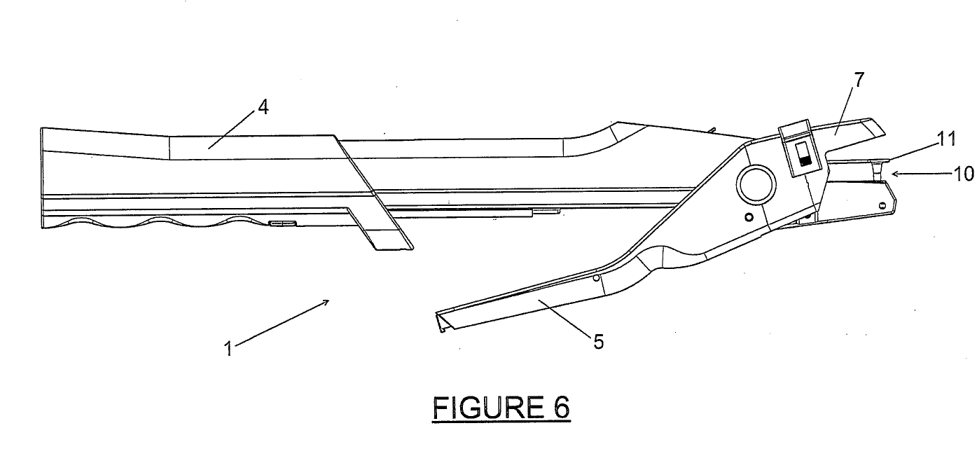

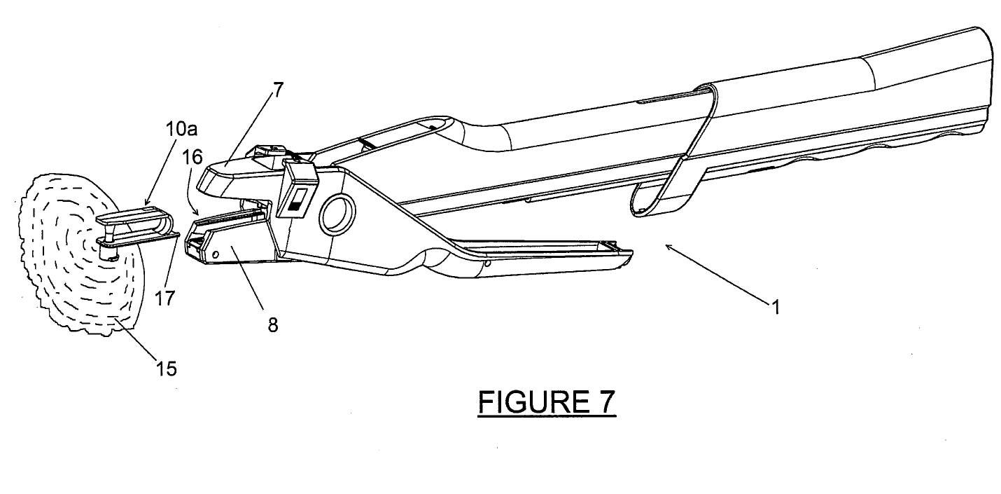

Then turning to FIGS. 5 and 6, pressure on the handle 5 has been reduced so that the top jaw portion 7 can lift away from the top surface of the male portion 11 of the tag 10, facilitating the removal of the tagged animal's ear from the applicator 1. Referring particularly to FIG. 7, the tag 10a is shown in position, very diagrammatically, on the animal's ear 15 and the chamber 16, defined between the upper and lower jaw portions 7 and 8, is now empty. Also as shown in FIG. 7, a rear edge 17 of the tag 10 has been cut so as to enable the release of the frontmost tag 10a, with the animal's ear, from the remainder of the strip of tags, which will still be positioned within the applicator 1. It is mentioned in this regard, however, that the applicator 1 of the present invention may, if required, enable only single tags to be inserted into the chamber 16 for an individual tagging operation, so as to avoid the necessity to load the applicator 1 with a full strip of tags when only a single tagging operation is necessary.

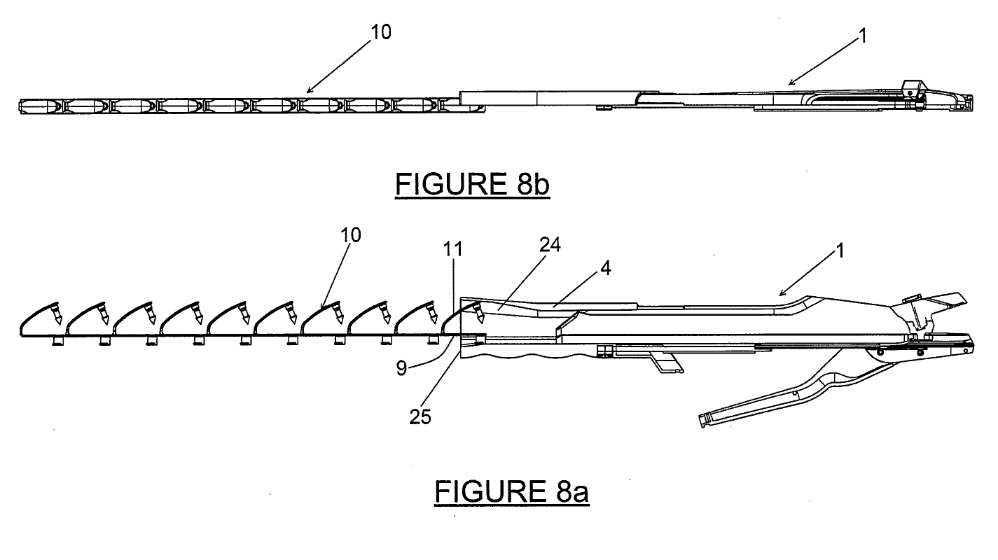

Turning then to FIGS. 8(a) and 8(b), the applicator 1 is shown, very diagrammatically, with a strip of tags 10 in the process of being loaded through the open end 25 of the rear body portion 4. As shown, the entrance 24 into the body portion 4 may be inwardly tapered so that as the tags 10 enter, the male portions 11 are caused to pivot or fold to some degree towards the female portions 9.

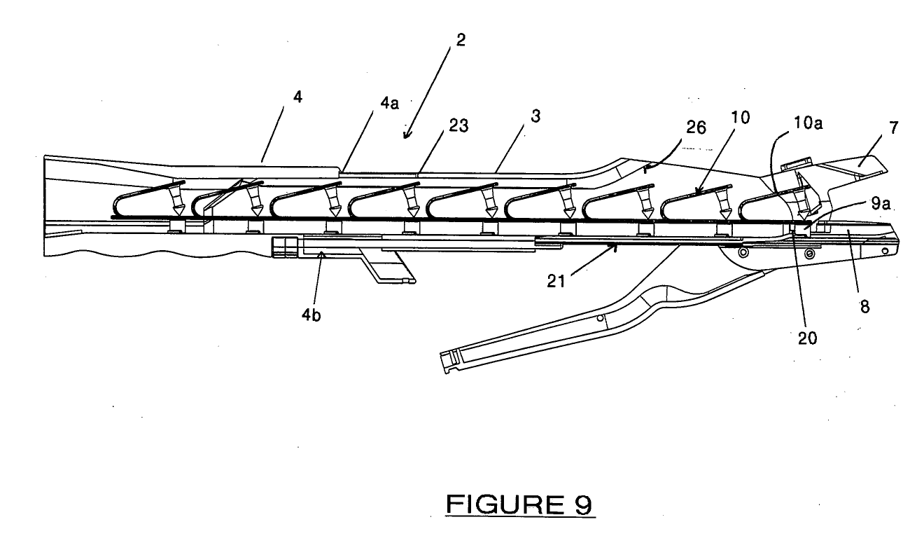

Referring then to FIG. 9, a strip of tags 10 is shown loaded in position, extending through the chamber defined by the substantially hollow body portions 3 and 4 of the applicator body 2. The frontmost tag 10(a), at this time is positioned behind the top jaw portion 7 and the lower jaw portion 8. Also in the position shown in FIG. 9, the rear body portion 4 is in its rearward position relative to the front body portion 3.

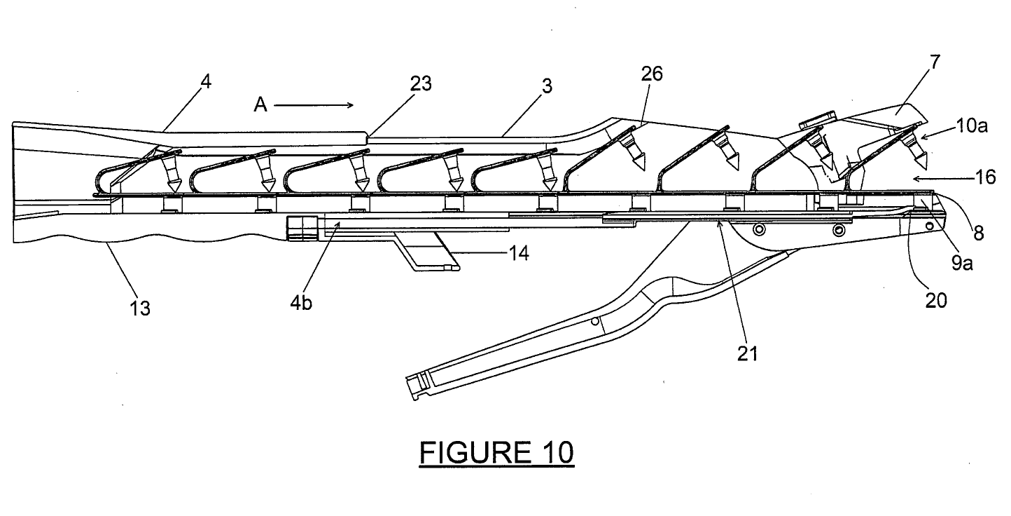

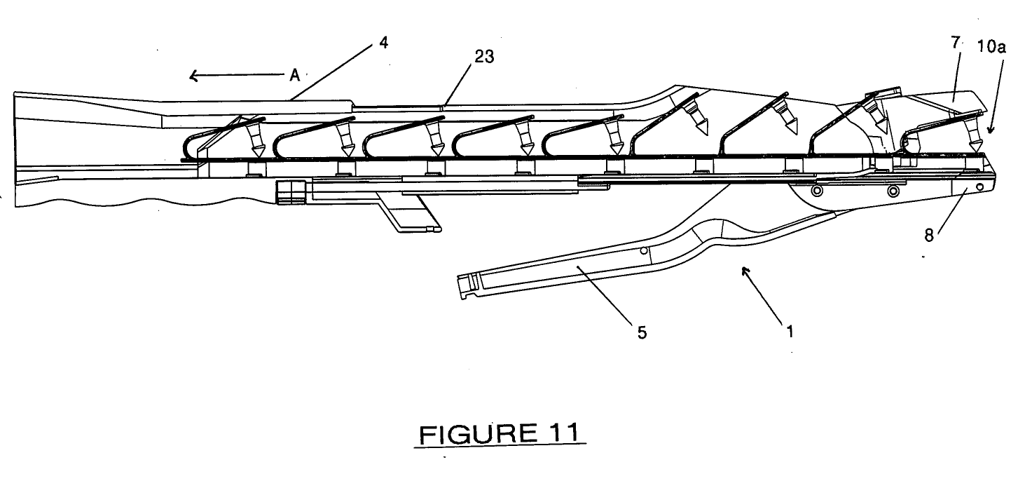

Turning then to FIG. 10, in this position the rear body portion 4 has been slid in the forward direction of arrow A. With the forward movement of the rear body portion 4, the frontmost tag 10(a) has now been pushed so that it is now positioned in the chamber 16 defined between the jaw portions 7 and 8. In positioning the frontmost tag 10(a) in the tagging chamber 16, (see FIG. 10) the slidable rear body portion 4 is moved forwardly so that its front edge 4(a) engages the abutment 23 on the rear edge of the front body portion 3 acting as a stop. In doing so, the bottom portion 4(b) of the body portion 4 pushes a feeder mechanism 21 forwardly so that a front end 20 of the feeder mechanism 21, which is engaged behind the rear of the frontmost female tag portion 9(a), pushes the frontmost tag 10(a) forwardly, see FIGS. 9 and 10 especially. It will also be noted from FIGS. 9 and 10 that the front body portion 3 provides a forwardly and upwardly inclined inner surface 26 making extra space available, enabling the front few tags 10 within that space to return to their uncompressed condition until they, in due course, become the frontmost tag 10(a), and are compressed between the jaw portions 7 and 8. The projection 14 and the finger grips 13 which facilitated the easy forward sliding movement of the rear body portion 4 into the position shown in FIG. 10 will now facilitate the return of the body portion 4 in the rearward direction of arrow A, into the position shown in FIG. 11, with the front end of the body portion 4 now shown spaced apart from the abutment 23. In FIG. 11, the handle 5 is also shown having been compressed by the user against its bias so that the jaw portions 7 and 8 have now moved towards their closed position in compressing the frontmost tag 10(a).

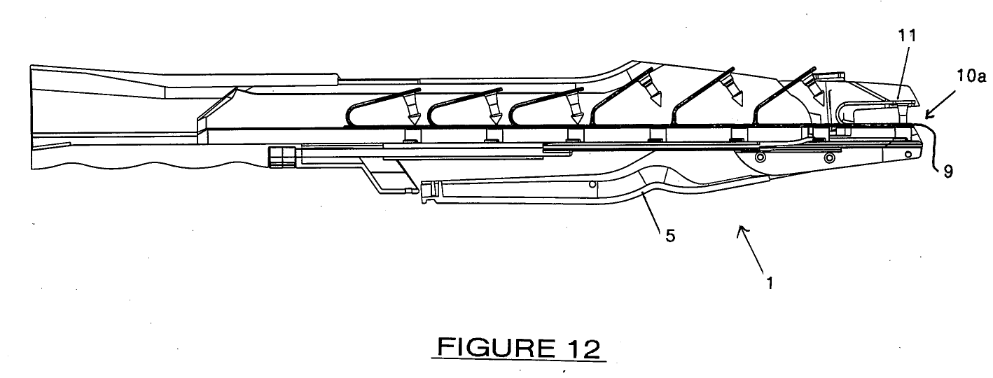

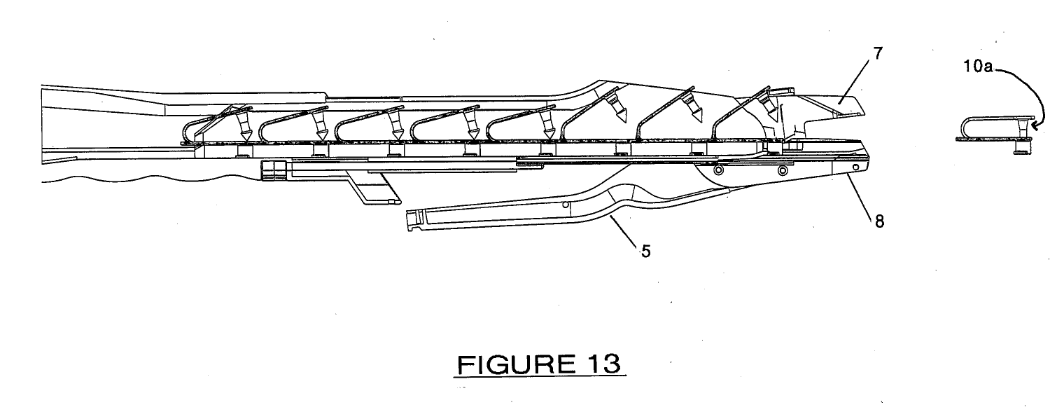

Turning then to FIG. 12, with the handle 5 fully compressed against its bias, the frontmost tag 10(a) has its male and female tag portions 11 and 9 fully engaged about the ear of the animal (not shown). Then, as shown in FIG. 13, with the pressure on the handle 5 reduced, the jaw portions 7 and 8 can separate allowing the release of the tag 10(a), now engaged about the animal's ear 15, (not shown), (see also FIG. 7).

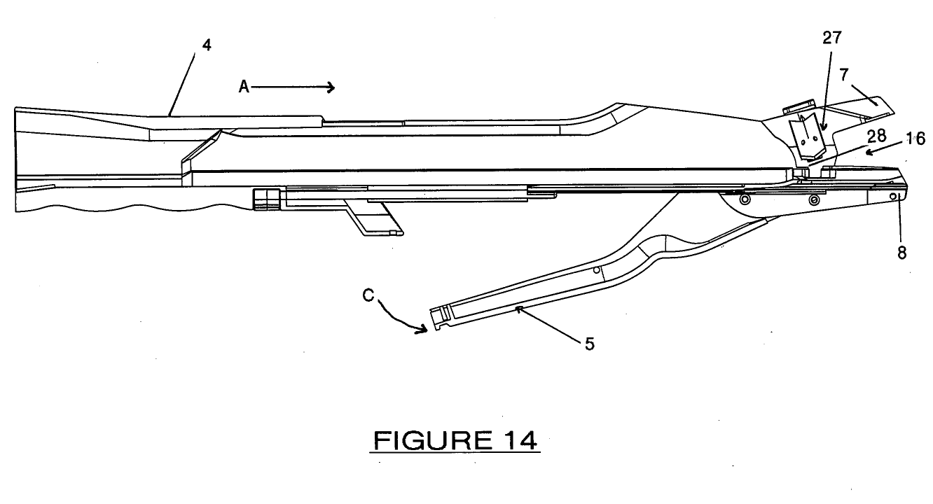

In FIG. 14, the handle 5 is shown having been allowed to be biased back into its open position, as indicated by the arrow C, by which time, with the chamber 16 between the jaw portions 7 and 8 now empty, the body portion 4 is ready to be slid forwardly in the direction indicated by arrow A to load the next frontmost tag (not shown) into position within chamber 16.

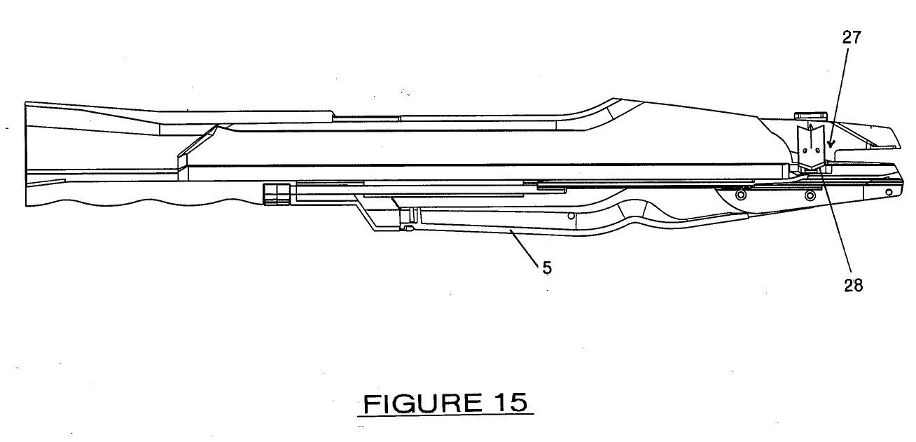

In FIGS. 14 and 15, a cutting means 27 is shown, very diagrammatically, moveable with the upper jaw portion 7 so that as the jaw portions 7 and 8 close together, a cutting edge 28 will engage behind the rear edge 17 of the frontmost tag 10(a), in separating it from the next following tag 10, (see FIG. 7).

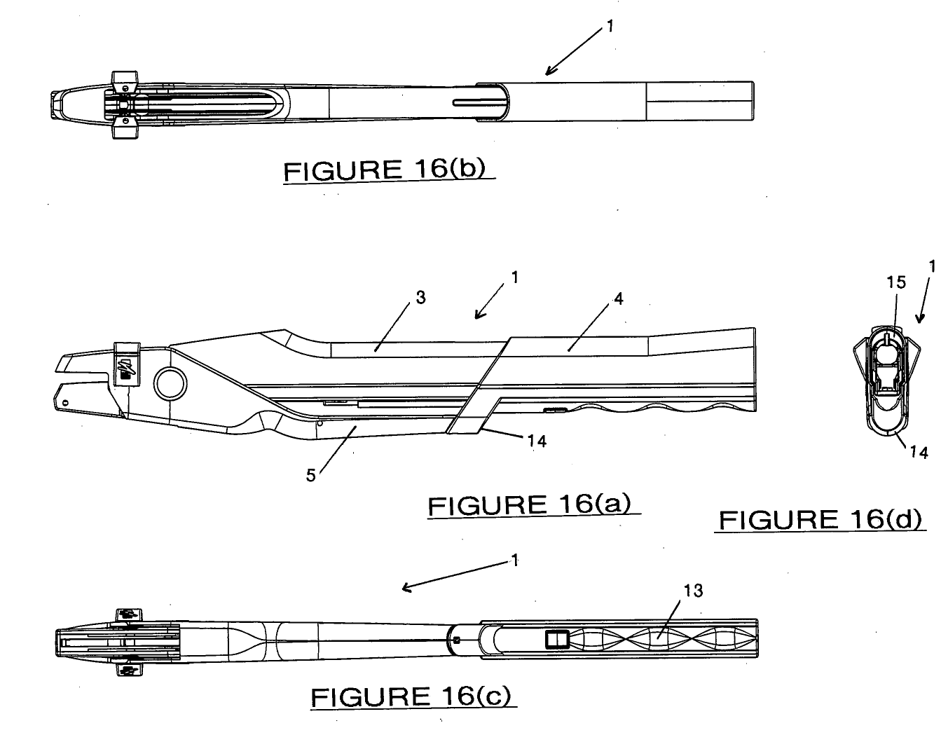

Referring now to FIGS. 16 (a), (b), (c) and (d), when the applicator 1 is not required for use, it may suitably be able to be stored in a storage condition with the handle 5 held in its closed position, see FIG. 16(a) especially, by being accommodated within the cavity provided by the projecting portion 14 of the rear body portion 4. When required for use, the body portion 4 may simply be slid rearwardly releasing the handle 5 to resume its biased open position.

It is thus seen that an ear tag applicator of the present invention can facilitate the simple yet effective loading of tags before sequential tagging operations and can reduce the effort required by a user of the applicator.

An additional advantage of the present invention is that the tag loading operation requires minimal interacting parts, which in the harsh environment of tagging operations have in the past with previous loading mechanisms caused frequent malfunctions.

Where in the foregoing description reference has been made to specific components or integers of the invention having known equivalents then such equivalents are herein incorporated as if individually set forth. Also where reference in the specification has been made to an “ear tag”, it is to be understood that this encompasses tags and applicators therefor which may be suitable for application to any part of an animal's body.

Although this invention has been described by way of example and with reference to possible embodiments thereof, it is to be understood that modifications and/or improvements may be made thereto without departing from the scope of the invention, as defined in the appended claims.

1. An animal tag applicator including an applicator body having at least first and second body portions defining a chamber adapted, in use, to accommodate a strip of tags substantially along a longitudinal axis of said body, said second body portion being adapted to slide over said first body portion in a substantially forward direction towards a front end of said first body portion whereby a frontmost tag of the strip of tags, in use, is located in a tagging position, and subsequently to slide in a substantially rearward direction over said first body portion away from said front end of said first body portion, before movement of the next tag into the tagging position, an operating means moveable relative to said first body portion and adapted, in use, to engage said frontmost tag for it to be secured about a part of an animal in a tagging operation.

2. (canceled)

3. An applicator as claimed in claim 1 in which said operating means includes a handle pivotally mounted relative to said first body portion, said handle is biased into an open position away from said first body portion and includes at a front end thereof, a first jaw portion movable towards a second jaw portion in engaging with said frontmost tag to provide said tagging operation.

4. An applicator as claimed in claim 3 in which the second jaw portion is provided at the front end of the first body portion and with said first jaw portion defines a further chamber in which said frontmost tag is located in said tagging position.

5. An applicator as claimed in claim 4 in which a biasing means biases said handle away from said first body portion, movement of said handle towards said first body portion against said biasing means moving said first jaw portion toward said second jaw portion in engaging said frontmost tag about said part of said animal in said tagging operation.

6. An applicator as claimed in claim 5 in which said second body portion, in sliding forwardly relative to said first body portion, causes the operation of a feeder means which is adapted to engage behind the frontmost tag and move it into said chamber between said jaw portions and into said tagging position.

7. An applicator as claimed in claim 6 in which said first body portion includes an abutment means to limit the forward movement of said second body portion.

8. An applicator as claimed in claim 3 in which said first jaw portion includes a cutting means which is adapted, with the movement of said first jaw portion toward said second jaw portion prior to or during a tagging operation, to engage with and cut a rear edge of said frontmost tag in separating it from a next following tag in said strip of tags.

9. An applicator as claimed in claim 1 in which said second body portion includes a substantially open rearward end adapted to accommodate the introduction therethrough of said strip of tags into said chamber defined by said first and second body portions.

10. An applicator as claimed in claim 3 in which said tags have male and female portions which are engaged together in said tagging operation and wherein said first jaw portion is adapted to receive said male portion and said second jaw portion is adapted to receive said female portion.

11. An applicator as claimed in claim 3 in which said second body portion includes a projecting portion adapted to be grippable by a user in sliding the second body portion forwardly and rearwardly over said first body portion.

12. An applicator as claimed in claim 11 in which said handle is adapted to be locatable within said projecting portion in a storage position.

13. (canceled)
